(19) United States
(12) Patent Application Publication
(10) Pub. No.: US 2018/0224376 A1
(43) Pub. Date: Aug. 9, 2018

(54) METHOD AND DEVICE FOR ANALYSING MOLECULAR INTERACTIONS, AND USES THEREOF

(71) Applicant: École Polytechnique / dgar, Palaiseau Cedex (FR)

(72) Inventors: Silvan TURKCAN, Paris (FR); Jean-Marc ALLAIN, Antony (FR); Antigoni ALEXANDROU, Palaiseau (FR)

(21) Appl. No.: 15/885,349

(22) Filed: Jan. 31, 2018

Related U.S. Application Data
(62) Division of application No. 13/811,377, filed on Apr. 1, 2013, now abandoned.

(30) Foreign Application Priority Data
Jul. 21, 2010 (FR) 1055940

(51) Int. Cl. G01N 21/64; G01N 33/58

(52) U.S. Cl. CPC G01N 21/6486; B01L 3/502761; G01N 33/582; G01N 33/587

(57) ABSTRACT

The invention relates to a method for analysing an interaction between a first molecule and a second molecule bonded to a particle, including the following steps: contacting the first molecule with the second molecule bonded to the particle under conditions enabling the interaction thereof; applying a predetermined liquid flow to the particle bonded to the second molecule; observing a movement of the particle bonded to the second molecule caused by the applied flow; analysing the interaction according to the movement observed and the applied flow, the particle having a greater hydrodynamic resistance than the first and/or second molecule, and a mass Péclet number of greater than 1. The invention also relates to a device for analysing an interaction between a first molecule and at least one second molecule, as well as to the use of the method or of the device in screening a candidate molecule for developing a drug.

### PRIORITY CLAIM

This is a divisional application of U.S. application Ser. No. 13/811,377, filed Apr. 1, 2013, which is a National Phase entry of PCT Application No. PCT/FR2011/051773, filed Jul. 21, 2011, which claims priority from French Application No. 1055940, filed Jul. 21, 2010, the disclosures of which are hereby incorporated by referenced herein in their entirety.

### FIELD OF THE INVENTION

The present invention relates to a method for analyzing an interaction between a first molecule and at least one second molecule bonded to a particle by means of the application of a force on said particle, and also to a device for analyzing this interaction and to the use of the method and of the device in screening for a candidate molecule for developing a medicament.

The present invention has applications in particular in the research field as a laboratory tool.

In the description below, the references between square brackets ([number]) refer to the list of references provided at the end of the text.

### BACKGROUND OF THE INVENTION

The high throughput measurement of biomolecular interactions, in particular for proteins but also for lower molecular weight molecules such as oligonucleotides, is a considerable challenge in certain fields of application such as research, diagnosis or screening to search for new drugs.

In order to measure these interactions, it is possible to measure, for example, the capacity and the kinetics of association between the biomolecules.

Among the techniques currently available, surface plasmon resonance is a widely employed technique which makes it possible to measure, without labeling, whether a protein present in solution interacts with ligands immobilized on a surface (Piehler et al., “New methodologies for measuring protein interactions in vivo and in vitro”, Curr Opin Struct Biol. 2005 (February; 15(1):4-14, [1]). This technique can also be carried out with a ligand fixed in an artificial lipid bilayer (Cooper M A, “Advances in membrane receptor screening and analysis”, J Mol Recognit. 2004 July-August; 17(4):286-315, [2]). The detection is based on the fact that the binding of the protein to the surface induces a modification of the refractive index. This leads to a shift in the surface plasmon resonance frequency, which is reflected either by a change in the reflected intensity at a fixed angle, or by a change in the angle at which the maximum reflected intensity is observed. One of the drawbacks of this technique is that the modification of the signal during the binding of small molecules is very low, and difficult to detect.

Solutions for studying interactions between molecules have been sought in the development of tools which make it possible, in particular in real time, either to exert a force on one of the two molecules, or to measure the force exerted or produced by small molecular structures.

Optical tweezers are thus a tool which makes it possible to nanomanipulate an object and, in particular, to apply a force to it without touching it, that is to say without there being any material contact with the object (S. Chu, Laser Manipulation of Atoms and Particles, Science 253, 861 (1991) ([3])). Optical tweezers use the minimum energy produced by a laser focused so as to be able to move a micrometric bead and thus apply a force to the objects attached to the bead. Thus, a bead attached to a membrane protein or a lipid has been trapped by optical tweezers in order to study its interaction with the membrane following the application of a force via the optical tweezers (A. Pralle, P. Keller, E.-L. Florin, K. Simons, and J. K. H. Hörber, Sphingolipid-Cholesterol Rafts Diffuse as Small Entities in the Plasma Membrane of Mammalian Cells, J. Cell Biol. 148, 997-1007 (2000), ([4]), Kenichi Suzuki, Ronald E. Sterba, and Michael P. Sheetz, Outer Membrane Monolayer Domains from Two-Dimensional Surface Scanning Resistance Measurements, Biophys. J. 79 448-459 (2000) ([5])).

Another way to manipulate a biomolecule is to use magnetic tweezers. For example, by specifically attaching a DNA molecule via its ends between a glass surface and a paramagnetic microbead, it is possible to mechanically manipulate this molecule by acting on the bead by means of a magnetic field produced by magnets.

Other tools for applying a force in order to study the interaction between molecules have been proposed: atomic force microscopy (AFM) (Moy, V. T., Florin, E.-L. & Gaub, H. E. Intermolecular forces and energies between ligands and receptors. Science 264, 257±259 (1994) ([6]), Hinterdorfer, P., Baumgartner, W., Gruber, H. J., Schilcher, K. & Schindler, H. Detection and localization of individual antibody-antigen recognition events by atomic force microscopy. Proc. Natl Acad. Sci. USA 93, 3477-3481 (1996) ([7])) and a technique known as “biomembrane force probe” based on the mechanical movement of a micropipette with a membrane capsule at the end, the membrane tension of which can be modulated via the suction pressure of the micropipette (Evans, E., Ritchie, K. & Merkel, R. Sensitive force technique to probe molecular adhesion and structural linkages at biological interfaces. Biophys. J. 68, 2580-2587 (1995) ([8]), R. Merkel, P. Nassoy, A. Leung, K. Ritchie, E. Evans, Nature 397, 50-53 (1999) ([9])).

The application of a force by means of optical or magnetic tweezers or by means of an AFM tip or of a micropipette involves complex processes, which require expensive material, in particular high-power lasers or magnets in the case of the tweezers, complex electronics in the case of AFM and difficult alignments in the case of the micropipette. In addition, the material used is bulky and not easy to move, which makes its use difficult or even impossible in a mobile kit. Moreover, in the case of optical tweezers, the energy of the lasers can be absorbed by the medium and dissipated as heat and can cause denaturation of the molecule to be studied. Furthermore, these techniques do not or do not readily lend themselves to multiplexing.

Methods have been developed for evaluating the interactions between biomolecules without using these devices, in particular lasers.

For example, document WO 2009098272 ([10]) describes the evaluation of the adhesion, movement and morphology properties of cells subjected to a flow. The device described is a device for the macroanalysis of a deposit of platelets or protein films on a surface. The device described in this document does not, however, make it possible to analyze interactions at the molecular level.

Document WO2006009398 ([11]) describes a method for detecting molecular interactions that can be used in vivo and in vitro, and also a microfluidic device for determining the interactions between two biomolecules, in which one of the biomolecules is labeled, and in which the biomolecules are brought into contact by means of a flow applied thereto. The drawback of this technique lies in the use of a magnetic field for exerting the force, which requires expensive equipment. Moreover, it does not make it possible to gain access to the dynamics of the interaction between biomolecules, since the application of the force and the detection are two distinct steps.

The document Schmidt Brian J. et al. “Catch Strip Assay for the Relative Assessment of Two-Dimensional Protein Association Kinetics”, Anal. Chem. 2008, 80:944-950 [49] has proposed analyzing the interaction between a first and a second molecule by virtue of the observed movement of a microparticle bonded to the second molecule and the application of a flow. In the case in which the particle is a microparticle, a large number of second molecules will be bonded to the microparticle. In this situation, it is only possible to observe the average between a large number of interactions between first molecules and second molecules. This approach does not make it possible to study a single pair of first molecule and second molecule. If there are, for example, two forms of first molecules, M1 and M1*, or two forms of second molecules M2 and M2*, this approach will not make it possible to distinguish differences in interaction between M1-M2 and M1*-M2 or between M1-M2 and M1-M2*. Likewise, this approach does not make it possible to study the interaction of a single first molecule with its environment following the application of a force via the second molecule. Indeed, the approach of the reference Schmidt Brian J. et al., Anal. Chem. 2008, 80:944-950 [49] makes it possible to study the interaction of a set of molecules M1 “of unknown number” with their environment. It is thus impossible to work back to the energy of interaction of a single molecule with its environment or to the distribution of values of the energy of interaction between two molecules.

Having this information is, however, essential for being able to understand the nature of these interactions, but also for effective screening for a candidate molecule for developing a medicament.

There is therefore a real need for a tool and for a method for analyzing an interaction between molecules which overcome these faults, drawbacks and obstacles of the prior art, in particular a tool of which the use is simpler, more practical, less expensive, compatible with a simultaneous study of multiple interactions and sufficiently precise for the analysis, on the molecular scale, of the interactions between molecules.

In particular, there is therefore a need for a tool and for a method for analyzing an interaction between molecules at the level of a single pair of molecules.

### SUMMARY OF THE PRESENT INVENTION

The present invention actually makes it possible to overcome the drawbacks of the prior art and to meet this need.

In some aspects, the present invention is directed at a method for analyzing an interaction between a first molecule and a second molecule bonded to a particle, comprising bringing the first molecule and the second molecule bonded to the particle into contact under conditions enabling the interaction thereof, applying a predetermined liquid flow to the particle bonded to the second molecule, observing a movement of the particle bonded to the second molecule caused by the applied flow, and analyzing the interaction according to the movement observed and to the applied flow, wherein the particle having a greater hydrodynamic resistance than the first and/or second molecule, and having a mass Péclet number greater than 1, and having a nanometric size dimension between 1 and 100 nm.

In some aspects, the movement of the particle is observed by means of the physical and/or chemical properties of the particle.

In some aspects, the observation is carried out by a method selected from the group comprising an optical method, a chemical method, an electrochemical method and a magnetic method.

In some aspects, the observation is carried out by an optical method selected from the group comprising the use of a fluorescence emitter and of a means for detecting fluorescence, detection of absorption, detection of reflection, detection of scattering and detection of diffraction.

In some aspects, the fluorescence emitter is the particle itself or a label associated with the particle and/or with at least one molecule selected from the first molecule and the second molecule.

In some aspects, the predetermined liquid flow is such that it makes it possible to apply to the particle a force F equal to 6πμRv, wherein μ represents the viscosity of the fluid, R the hydrodynamic radius of the particle and v the speed of the fluid around the particle.

In some aspects, the analysis is qualitative or quantitative.

In some aspects, the interaction is an interaction between biological molecules or between a biological molecule and a chemical molecule.

In some aspects, the first molecule is a molecule of a cell membrane.

In some aspects, the present invention is directed at a device for analyzing an interaction between a first molecule and at least one second molecule, the device comprising a means of applying a predetermined liquid flow, a support for receiving a first molecule, a particle capable of bonding a second molecule capable of interacting with the first molecule, said particle having a nanometric size dimension between 1 and 100 nm, and a means for observing the movement of the particle, in which the particle has a greater hydrodynamic resistance than the first and/or second molecule, and a mass Péclet number of greater than 1.

In some aspects, the means for applying the predetermined liquid flow is a means which makes it possible to apply to the particle, via the liquid flow, a force F equal to 6πμRv, in which μ represents the viscosity of the fluid, R the radius of the particle and v the speed of the fluid around the particle.

In some aspects, the means for observing the movement of the particle is a means of observation using physical and/or chemical properties of the particle.

In some aspects, the present invention is directed to the foregoing method of the present invention or the foregoing device of the present invention, in screening for a candidate molecule for developing a medicament.

### BRIEF DESCRIPTION OF THE FIGURES

FIG. 1A represents the geometry of the microfluidic channel, which comprises an inlet for culture medium (EM), an inlet for nanoparticle solution (ENP), an outlet (S) and a measurement zone between them.

FIG. 1B illustrates a method of measurement by means of a microscope, in which a flow (f) is applied to the nanoparticle (NP) and the second molecule (M2), bound to the first molecule (M1). The second molecule (M2) consists of a peptide toxin bound to its receptor. The first molecule (M1) consists of the cell membrane and also all the elements of the cell (including the cytoskeleton) with which the toxin receptor interacts. The application of the predetermined flow (f) to the particle (NP) creates a hydrodynamic force on the particle (NP); this force is transmitted to the second molecule (M2) and makes it possible to observe the effect of this force on the interaction between the first molecule (M1) and the second molecule (M2).

FIG. 2A represents the movement (Dp) in μm of the receptor of the toxin protein at the surface of the cell membrane as a function of time (t, in seconds) following the application of a flow (f) with a flow rate of 7.5 μl/min between t=95 and 128 s. The position of the receptor is visualized by means of the fluorescence of the nanoparticles. The receptor moves in the direction of the flow (f) and then stabilizes at a new position shifted by approximately 2 μm relative to its initial position before application of the flow (f). The movement in the direction perpendicular to the direction of the flow (f) is negligible compared with the movement in the direction of the flow (f). After the flow (f) is stopped, the receptor goes back to a position close to the initial position. This return to a position close to the initial position implies that the receptor is attached to the cytoskeleton of the cell. The equilibrium position under flow (f) will then correspond to the position for which the force applied to the receptor via the hydrodynamic force on the particle is equal to the return force of the cell. When the flow (f) is stopped, only the return force of the cell acts on the receptor, which goes back to the initial position with a speed v, determined by Stokes' law: F=γv, where γ is the drag coefficient of the receptor in the system made up of the membrane and the cytoskeleton, and F is the force due to the flow (f). Knowing v and F, it is thus possible to determine the friction coefficient.

FIG. 2B (top) represents the movement (Dp, in μm) as a function of time (t, in seconds) of the receptor relative to its initial position following the application of a succession of flows (f, in μl/min) of different flow rates (bottom). The equilibrium positions with and without flow are shown. The dashed rectangle indicates the part of the experiment which is shown in FIG. 2A.

FIG. 3 represents the average movement (Dp, in μm) as a function of the force applied by means of the flow (f), the flow rate (De) of which is expressed in μl/min, obtained from experiments carried out under the same conditions as those of FIG. 2B with a sequence of different flow rates (De). The movement of the toxin receptor is linearly dependent on the applied force. As indicated, the equilibrium position under flow corresponds to the position for which the force applied to the receptor via the hydrodynamic force on the particle is equal to the return force due to the cell. The return force of the cell can be modeled by means of Hooke's law, F=k·x, where k is the stiffness constant and x the movement relative to the equilibrium position. This modeling by means of Hooke's law makes the hypothesis that the deformation of the cell is elastic and the movement of the receptor is reversible. The toxin receptor actually goes back to a position close to the initial position. The linear adjustment of the experimental data of FIG. 3 makes it possible to determine the stiffness constant k.

FIG. 4 represents the movement (Dp, in μm) of the receptor as a function of time (t, in seconds) relative to its initial position following the application of a succession of flows (f, in μl/min) of different flow rates (indicated by a dashed line). Only the equilibrium positions with and without flow are shown in this figure. Several biomechanical effects can be identified during this experiment. An elastic deformation state is observed, where the receptor goes back to its initial position after the flow (f) is stopped. After application of a flow (f) with a flow rate of 30 μl/min, the receptor does not go back to its original position after the flow (f) is stopped: this is a plastic deformation state. Finally, the application of a flow (f) with a flow rate of 50 μl/min causes detachment of the toxin from its receptor. The observation of a certain number of detachment events can be used to determine the dissociation constant k_{off} between the toxin and its receptor, as explained in the description of the invention.

FIG. 5 represents an image of confluent MDCK (Madin-Darby canine kidney) cells obtained by white light transmission. The paths of 4 receptors obtained, represented by the position X (PX in μm) and the position Y (PY in μm) following the application of a succession of flows with different flow rates, as in FIG. 2B, are indicated in black lines. This figure illustrates the multiplexing capacity of this technique.

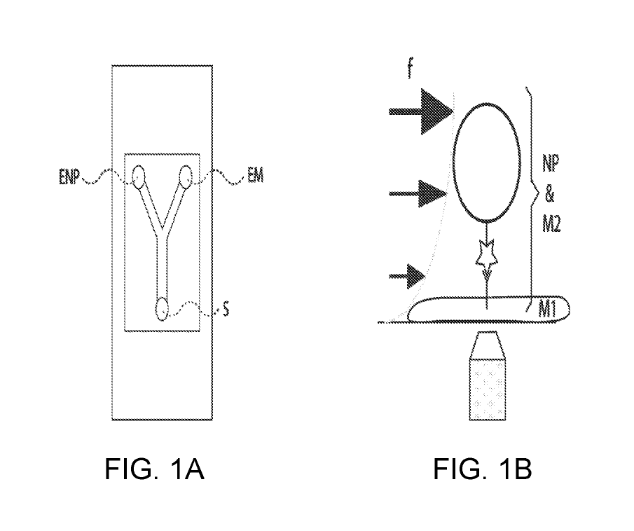

### DESCRIPTION OF THE PRESENT INVENTION

Following considerable research, the applicant has developed a method for analyzing an interaction between molecules, the implementation of which is simple, inexpensive, practical, and sufficiently accurate to analyze, on the molecular scale, interactions between molecules. In particular, the method of analysis does not require the use of a laser or of a magnet or of an AFM tip to generate a force, nor of a complex instrument for reading the results. It also makes it possible to analyze interactions between molecules without damaging them or denaturing them. The present invention also makes it possible to analyze the interaction of a very small molecule with a ligand, or of very small molecules with one another. Furthermore, the present invention makes it possible to study a low number of interactions, or even a single interaction, between a first molecule and a second molecule. It also has a capacity for multiplexing and makes it possible, in particular, to simultaneously and individually study a large number of interactions between single pairs of first and second molecules.

Thus, a first subject of the present invention relates to a method for analyzing an interaction between at least one first molecule and at lea3340st one second molecule bonded to a particle, comprising the following steps:
bringing the first molecule into contact with the second molecule bonded to the particle under conditions enabling the interaction thereof,
applying a predetermined liquid flow to the particle bonded to the second molecule,
observing a movement of the particle bonded to the second molecule caused by the applied flow,
analyzing the interaction according to the movement observed and to the applied flow,
   the particle having a greater hydrodynamic resistance than the first and/or second molecule, and a mass Péclet number of greater than 1.

Another subject of the present invention relates to a device for analyzing an interaction between a first molecule and at least one second molecule, comprising: a means for applying a predetermined liquid flow, a support for receiving a first molecule, a particle capable of bonding a second molecule capable of interacting with the first molecule and a means for observing the movement of the particle, wherein the particle has a greater hydrodynamic resistance than the first and/or second molecule, and a mass Péclet number of greater than 1.

In the present description, “a molecule” means “at least one molecule”, i.e. one or more molecules. When it is a question of several molecules, they may be identical or different. The first and the second molecule may be identical or different. This may also involve a first molecule M1 present in two or more different forms, for example M1 and M1*, or a second molecule M2 present in two or more different forms, for example M2 and M2*. In this precise case, it will be a question of detecting and comparing, during the same experiment, the affinity of a single first molecule M1 for a single second molecule M2 compared with a second molecule M2* or detecting and comparing, during the same experiment, the affinity of a single second molecule M2 for a single first molecule M1 compared with a first molecule M1*.

It may be an organic or inorganic molecule. When it is an organic molecule, it may be a synthetic molecule or a molecule of biological origin. In the case of molecules present in two or more different forms, M and M*, it may be, for example, two or more different conformations of the same protein or two or more different types of DNA helices.

In other words, the present invention may advantageously be used for the analysis of any type of molecular interaction, and therefore for any type of molecules that interact. It may be an interaction of organic/organic, organic/inorganic or inorganic/inorganic type.

For the purpose of the present invention, the term “molecular interaction” is intended to mean any noncovalent and reversible bond. The interaction may be specific or nonspecific. In the case of a specific interaction, it may be, for example, an antigen-antibody, ligand-receptor or enzyme-substrate interaction, this list not being limiting. In the case of a nonspecific interaction, it may be, for example, an interaction between a biological molecule and a surface.

When the first molecule and/or the second molecule is (are) an organic molecule, it (they) may be selected, for example, from the group comprising an amino acid, a peptide, a protein, a nucleoside, a nucleotide, a nucleic acid, a fatty acid, a simple or complex lipid, a sugar, a monosaccharide, a polysaccharide, a phospholipid, a glycolipid, a glycoprotein, a lipoprotein, a glycolipoprotein, or any other molecule of biological origin, which interacts or is capable of interacting with another molecule. When the molecule is a nucleic acid molecule, it may be DNA (deoxyribonucleic acid) or RNA (ribonucleic acid). When the molecule is a peptide or protein molecule, it may be a molecule chosen from the group comprising a candidate medicament, a molecule of a cell membrane, a toxin, an antibody, a matrix component of the skin, for instance collagen or fibronectin, or a receptor, in particular a membrane receptor. It may also be a question of multimolecular systems, for example a ribosome, a proteosome, an apoptosome, a centrosome, or cell compartments such as a mitochondrion, an endosome, a lysosome, a phagosome, a vesicle, or even also a cell, a single-cell organism, for instance a bacterium, a yeast, a fungus, a single-cell alga or a protozoan. It may also be a multicellular system, for instance a cell culture, an epithelium, a tissue fragment or a bacterial colony.

When the molecule is an inorganic molecule, it may be any inorganic molecule known to those skilled in the art or an inorganic surface, for instance a polymer surface, a metal surface, a fluorinated surface or a silicon surface. By way of example, the first molecule may be a cell membrane receptor, and the second molecule may be a ligand of the receptor or a molecule capable of binding to this receptor.

The first or second molecule may advantageously have a molecular weight ranging from 300 to 300 000 g·mol⁻¹. Advantageously, it may be a small molecule, the molecular weight of which ranges from 100 to 4000 g·mol⁻¹.

The first molecule may be bonded to a receiving support or be a molecule of the receiving support. The receiving support may be any surface which makes it possible to receive the first molecule. Preferably, this surface allows the attachment of the first molecule thereto, preferably while preserving the conformation of the molecule. This is because, for example, when it is a protein that must interact with the second molecule, it is important for the zones of interaction of the protein with the second molecule not to be deformed by the attachment of the protein to the surface. Advantageously, the first molecule is bonded to the support sufficiently strongly so as not to be detached from the support by the predetermined liquid flow during the implementation of the method of the invention.

The attachment of the first molecule may be direct or indirect. It is direct when the first molecule interacts directly with the surface, either because the surface has been functionalized beforehand, or because it has intrinsic chemical properties which make it possible to attach the first molecule. It is indirect when a spacer arm or a multimolecular structure, for example a cell wall fragment, is attached to the surface.

Advantageously, the support may comprise one or more bonding zones on which the first molecule is bonded. When the support comprises a single bonding zone, one or more types of first molecule can be bonded to the zone. When the support comprises several bonding zones, each zone may comprise one or more types of first molecule. Advantageously, the first molecule may be different from one zone to another. Several bonding zones may be produced using a microdispenser (also called spotter) (I. Barbulovic-Nad, M. Lucente, Y. Sun, M. Zhang, A. R. Wheeler, M. Bussmann, Bio-Microarray Fabrication Techniques—A Review, Critical Reviews in Biotechnology, 26:237-259, 2006 ([12])) which enables the depositing of various molecules in controlled positions on a surface.

Advantageously, the support may make it possible to carry out several methods of analysis simultaneously or sequentially, for example with various types of particles and/or various types of first molecule and/or various types of second molecule and/or of first molecules slightly different than one another and/or of second molecules slightly different than one another.

The support may be a support of the type of those used in DNA chips, or in protein chips, this list not being limiting. They may, for example, be commercially available supports, such as the supports sold by Affymetrix (http://www.affymetrix.com/estore/).

Any material known to those skilled in the art which makes it possible to obtain these characteristics can be used, for instance glass, silicon, plastic, such as polystyrene, polydimethylsiloxane or polymethacrylate, this list not being limiting. The support may be fabricated according to any method known to those skilled in the art. The supports may, for example, be glass supports, for example those sold by VWR (http://fr.vwr.com/app/Home).

It may also be a transparent or opaque surface.

For the purpose of the present invention, the term “particle” is intended to mean any assembly of atoms forming an organic or inorganic object. The particle makes it possible to apply a force to the second molecule. Advantageously, the particle may be observed or visualized, which makes it possible to simultaneously observe or visualize the position or the movement of the second molecule.

For the purpose of the present invention, the term “hydrodynamic resistance” is intended to mean the force resulting from all the stresses exerted by the flowing of the predetermined liquid flow on the particle. The dimensions of the particle are such that it has a greater hydrodynamic resistance than the first and/or the second molecule. Advantageously, the hydrodynamic resistance of the particle can allow movement of the particle and of the second molecule, caused by a controlled liquid flow.

For the purpose of the present invention, the term “mass Péclet number” is intended to mean the ratio between the characteristic time of diffusion of a mass and the time of convection of this mass. It is equal to vL/D, where v is the characteristic speed of the flowing, L the characteristic size of the particle and D the diffusion coefficient of the particle. The mass Péclet number of the particle is much greater than 1. It may, for example, be between 1 and 10 000. These characteristics of the mass Péclet number imply that the particle has a sufficient size for its movement due to Brownian motion to be negligible compared with the effect of entrainment by the predetermined flow.

The particle may be a microparticle, i.e. have at least one dimension of micrometric size. For example, the microparticle has a size of between 1 and 100 μm, for example between 10 and 80 μm, or else between 20 and 70 μm, or between 30 and 50 μm.

Advantageously, the particle may be a nanoparticle, i.e. have at least one dimension of nanometric size. For example, the nanoparticle has a size of between 1 and 500 nm, for example between 1 and 200 nm, or else between 1 and 100 nm, for example between 10 and 80 nm, or else between 20 and 70 nm, or between 30 and 50 nm.

The particle may be any particle known to those skilled in the art, for instance glass, latex, polystyrene or metal particles, for example carbon, gold, silver, copper, and particles made of magnetic materials, this list not being limiting.

It may also be a nanoparticle or microparticles, of natural or synthetic origin, for instance a metal, a semiconductor, a metal oxide, a polymer or a nanosphere, this list not being limiting.

It is generally accepted in the nanoparticle user community that the aqueous medium surrounding a particle attached to a cell membrane molecule has no effect on its movement. In the absence of controlled movement of the fluid, it has been shown that the movement of the nanoparticle is not controlled by the surrounding fluid but by the behavior of the object to which it is attached. This is because, since the viscosity of the membrane is much greater than the viscosity of water, and despite the larger size of the nanoparticle compared with the size of a membrane molecule (membrane lipid or protein), the movement of the whole is determined by the movement of the membrane molecule.

Moreover, it is not easy to predict the hydrodynamic force exerted on a nanoparticle and a rapid calculation of the order of magnitude based on Poiseuille's and Stokes' law, given the fact that the speed is zero at the wall and that the nanoparticle and its system of attachment are generally very small in the face of all the other flowing scales, indicates forces that are too weak to have a significant effect on the interaction between the first and the second molecule or on the behavior of the first molecule. Contrary to what these facts imply, it is possible to exert a force, by means of a nanoparticle in a flow, that is sufficiently high to, for example, move a membrane molecule inside the cell membrane. Indeed, close to the surface of the channel, Stokes' law is no longer valid and the hydrodynamic forces exerted are greater than those predicted by Stokes' law: see A. J. Goldman, R. G. Cox, H. Brenner, Slow viscous motion of a sphere parallel to a plane wall—I Motion through a quiescent fluid, Chem. Eng. Sci. 22 637-651 (1967) ([16]), A. J. Goldman, R. G. Cox, H. Brenner, Slow viscous motion of a sphere parallel to a plane wall—Il Couette flow Chem. Eng. Sci. 22 653-660 (1967) ([17]), E. Lauga, T. M. Squires, Brownian motion near a partial-slip boundary: A local probe of the no-slip condition, Phys. Fluids 17, 103102 (2005) ([18]), J. Happel and H. Brenner, Low Reynolds Number Hydrodynamics, p 286-340 (Martinus Nijhoff, The Hague, 1983) ([19]).

The size of at least one of the dimensions of the particle may be greater than the size of the second molecule. The particle may have at least one dimension which is between five and one hundred times that of the second, for example between ten and twenty times.

The particle may have a shape suitable for being entrained by the predetermined liquid flow. The particle may have a substantially spherical, for example substantially round or oval, shape. It may in this case be a bead. Alternatively, the particle may have a nonspherical, for example ovoid, cylindrical or discoid, shape.

The particle may be any suitable molecule that is commercially available, for example from Invitrogen for the semiconductor-based Quantum Dots (http://www.invitrogen.com/site/us/en/home/brands/Molecular-Probes/Key-Molecular-Probes-Products/Qdot.html), or from British Biocell International for gold nanoparticles (http://www.britishbiocell.co.uk/products/goldreagents.asp?navid=2), or be manufactured by any means known to those skilled in the art, for example, for rare-earth-doped nanoparticles, according to the method described by Huignard et al. (Huignard, A.; Gacoin, T.; Boilot, J.-P. Chem. Mat. 2000, 12, 1090 ([13])).

Preferably, the bond between the particle and the second molecule is sufficiently stable so as not to be broken by the predetermined liquid flow. The particle and the second molecule can be bonded by any means known to those skilled in the art, for example via a weak chemical bond, for instance by electrostatic force, Van der Waals force, hydrogen bond, hydrophobic bonds, or else via a strong chemical bond, for instance via an ionic, covalent or metal bond, or else by means of a coupling agent, for instance coupling agents bearing double functions which make it possible to attach, on the one hand, to functions (for example amine functions or carboxylic acid functions) present at the surface of the particle and, on the other hand, to functions of the second molecule (for example, amine functions or sulfhydryl functions). Such coupling agents are commercially available, for instance from Pierce (http://www.piercenet.com/Products/Browse.cfm?fldID=0203). The particle and the second molecule can also be bonded by using, for example, strong-affinity biological interactions, such as the biotin-streptavidin interaction (or the ligand-receptor interaction or the antibody-antigen interaction), and multistep coupling, i.e. firstly coupling of the streptavidin (or of the biotin) to the particle and coupling of the biotin (or of the streptavidin) to the second molecule and then interacting with the two coupling products.

The first and the second molecule can be brought into contact in the analyzing device of the invention. The device of the invention may, in this regard, comprise any means for bringing the first and the second molecule into contact. It may be a channel with a geometry suitable for bringing the first and the second molecule into contact. The geometry of the channel may, for example, be straight, T-shaped or Y-shaped, or else be a more complex geometry in order to be able to add reactants independently. The geometry may be readily determined by those skilled in the art depending on the number of reactants introduced, or on the number of different first and/or second molecules introduced.

Advantageously, the device may have at least one inlet, so as to be able to introduce therein the first molecule, the second molecule bonded to the particle, and the predetermined liquid flow. The device may also have as many inlets as is required by the number of different first and/or second molecules injected into the device, or as many inlets as is required by the number of analyses simultaneously or sequentially carried out. For example, the device may have two inlets: one for the injection of the particles coupled with the second molecule, and the other for applying the liquid flow. The presence of two distinct inlets can make it possible to obtain a flow which does not contain particles bonded to the second molecule. The presence of two distinct inlets can thus make it possible to increase the signal/background noise ratio compared with a device with a single inlet.

Advantageously, the device may also comprise an outlet. The outlet may be connected to a reservoir. The reservoir may make it possible to modify the direction of the flow without the risk of introducing bubbles into the device. The reservoir is advantageously required only in the embodiments involving a change of direction of the flow.

The dimensions of the channel may be chosen according to the nature and the size of the first and/or of the second molecule and/or of the particle. For example, the channel may have a height, i.e. a distance between the receiving support of the first molecule and the upper wall of the channel, which is sufficiently high to allow the study of the interaction of the first and of the second molecule. The height may therefore depend on the dimensions of the first and/or of the second molecule. For example, when the first molecule is a cell, the consequence of a channel of insufficient height may be cells that are too confined and a negative impact on their physiology. The height of the channel is limited by the desired range of speed of the fluid at the level of the particle as a function of the means for controlling the flowing that are used: the higher the channel, the lower the speed. By way of example, the channel may have a height of between 20 and 70 μm, for example approximately 30 μm, or else approximately 40 μm, approximately 50 μm, or approximately 60 μm. For example, if the first molecule is a cell, the height of the channel may be approximately 50 μm. The width of the channel should be sufficiently large to make it possible to observe the desired amount of the first molecule. Advantageously, the height/width ratio should be sufficiently small to allow the creation of a predetermined liquid flow, corresponding to a laminar flow. The ratio may, for example, be between 1/100 and 1/2. The channel may have a width of between 100 μm and 2 mm, for example approximately 500 μm. The length of the channel may be sufficient to avoid disruptions in the predetermined liquid flow when the latter is subjected to an input and/or to an output of liquid. Advantageously, the length may be sufficient to provide hydrodynamic profiles of stream lines parallel to the walls of the channel over a sufficiently long zone of the channel. The channel may have a length of between 1 mm and 10 cm, for example approximately 1.5 cm. The length may make it possible to bind a sufficiently large number of first molecules to allow the analysis of the interaction. The length of the channel may be limited in cases where it lies on an optical device, for example on an optical plate.

The conditions for bringing the first and second particle into contact can be easily determined by those skilled in the art according to the first and second molecule.

For the purpose of the present invention, the term “liquid flow” is intended to mean any flowing of liquid. Advantageously, the flow makes it possible to apply a force to the particle. It can also make it possible to entrain the particle. The flow may be unidirectional or have several directions, which are for example alternating. Advantageously, the flow may be laminar. It may be liquid flowing of a solution, for example an aqueous solution, a saline aqueous solution with a pH fixed by a phosphate buffer (PBS) or by a HEPES buffer, a cell culture medium or any other solution required for studying the first and/or the second molecule under good conditions, or of a dispersion. Advantageously, the predetermined liquid flow can contain objects, floating in the flow and moved by the flow. Advantageously, measuring the speed of these objects can make it possible to measure the speed of the fluid around the particle. This measurement can be carried out in a step separate from that of the analysis of the interaction between first and second molecule.

For the purpose of the present invention, the term “predetermined flow” is intended to mean any flow of which the characteristics are such that they make it possible to control the force applied to the particle. In other words, the predetermined flow may be a flow of which the characteristics make it possible to select the amplitude and the direction of the force applied to the particle. Advantageously, the predetermined flow is such that it can make it possible to apply a force to the particle that causes the movement of said particle. The force is such that it does not cause detachment of the first molecule from the support nor any damage to the support. The characteristics of the flow can be determined by means of controlling at least one of the parameters chosen from the group comprising the flow rate or the speed of the liquid, the viscosity of the liquid, and the pressure applied to the liquid.

The viscosity of the liquid can be determined by any method known to those skilled in the art, for instance using a capillary viscometer by measuring the flow rate generated by a difference in pressure at the two ends of a capillary tube (Poiseuille's law) or using a Couette-Plate viscometer (P. Cousson, C. Ancey, Rhéophysique des pâtes et des suspensions [Rheophysics of pastes and suspensions], Ed. EDP Sciences (1999) ([14]).

The speed of the predetermined liquid flow may be continuous, i.e. have a constant value. Alternatively, the speed may be discontinuous, i.e. have several values. In this case, the speed may increase or decrease linearly. Alternatively, the speed may increase or decrease in steps, with periods between each step that may be easily determined by those skilled in the art depending on the information that they desire to collect.

The speed of the predetermined liquid flow can be measured by any method known to those skilled in the art. Advantageously, the speed of the predetermined liquid flow can be determined easily at each instant, by means of a method known to those skilled in the art, for instance a calculation of the speed as a function of the distance from the wall according to Poiseuille's law. If it is an electro-osmotic flow, the speed is constant within the channel and depends on the electric field applied and on the geometry of the channel (P. Tabelling, Introduction à la microfluidique [Introduction to microfluidics], p 178-202 Editions Belin (2003) ([15])). The speed of the predetermined liquid flow at the position of the particle can be calculated from the flow rate and/or from the speed of the flow upstream and/or downstream of the particle. In this case, the particle is to modify the flow only weakly and not to influence the other particles and the measurement of the flow rate. This calculation method also makes it possible to make quantitative comparisons between several experiments. Alternatively, the speed can be measured by visualizing unbound floating markers which do not interact with the first and/or the second molecule, and which are transported by the flow. Various floating markers, for example of various sizes, can be injected into the flow. For example, fluorescent beads can be used for this purpose.

The predetermined liquid flow may be such that it makes it possible to apply to the particle a force F equal to 6πμRv, in which μ represents the viscosity of the fluid, R the hydrodynamic radius of the particle and v the speed of the fluid at the position of the particle, or a force F equal to 6πμRv and corrected for the effects of proximity of a wall. The speed of the fluid can be measured upstream or downstream of the particle, at a distance which is large considering the particle, but small considering the other modifications of the flow. In the case of a solid spherical particle of a few tens of nanometers, its radius is very close to its hydrodynamic radius. In the case of a particle of very small size or of a nonspherical particle, its hydrodynamic radius can be determined, for example, by dynamic light scattering. Instruments for measuring the hydrodynamic radius of particles or of molecules by dynamic light scattering are sold, inter alia, by the company Malvern (http://www.malvern.com/zetasizer). When the particle is located at a distance from the channel wall that is comparable to its hydrodynamic radius, corrective factors linked in particular to the distance from the particle to the wall, the geometry of the particle and the characteristics of the flow (viscosity, speed) can be applied to the above formula, for example according to the techniques mentioned in the following documents: A. J. Goldman, R. G. Cox, H. Brenner, Slow viscous motion of a sphere parallel to a plane wall—I Motion through a quiescent fluid, Chem. Eng. Sci. 22 637-651 (1967) ([16]), A. J. Goldman, R. G. Cox, H. Brenner, Slow viscous motion of a sphere parallel to a plane wall—II Couette flow Chem. Eng. Sci. 22 653-660 (1967) ([17]), E. Lauga, T. M. Squires, Brownian motion near a partial-slip boundary: A local probe of the no-slip condition, Phys. Fluids 17, 103102 (2005) ([18]), J. Happel and H. Brenner, Low Reynolds Number Hydrodynamics, p 286-340 (Martinus Nijhoff, The Hague, 1983) ([19]).

The predetermined liquid flow can be applied, and its parameters controlled, by any means known to those skilled in the art, for example any device used in microfluidics for controlling flowing. The flowing can, for example, be applied via a syringe mounted on a syringe driver, via a pressure-drum device or via a peristaltic pump.

For the purpose of the present invention, the term “movement” is intended to mean any change of spatial position of the particle relative to the moment at which the first and second molecule interact. Advantageously, the position of the particle is measured at the moment at which the first and second molecule interact, and optionally at the moment at which the predetermined liquid flow begins to be applied. Advantageously, the position of the particle can be measured at regular intervals before and after the application of the predetermined liquid flow. Advantageously, the movement of the particle may be accompanied by the movement of the second molecule. Advantageously, the movement of the particle may cause breaking of the interaction between the first and the second molecule.

The movement or the position of the particle can be observed by virtue of the physical and/or chemical properties of the particle. The observable physical properties of the particle may be its absorption, its reflection or its scattering of light, its fluorescence, or its magnetic or electrical properties. The chemical properties of the particle may, for example, give rise to a reaction with a substrate resulting in the generation of a fluorescent or absorbent product. This may involve chemiluminescence or bioluminescence.

The movement of the particle bonded to the second molecule caused by the applied flow can be observed by any means known to those skilled in the art. It may, for example, be a technique chosen from the group comprising an optical method, a chemical method, an electrochemical method and a magnetic method (Jin-Woo Choi, Kwang W. Oh, Jennifer H. Thomas, William R. Heineman, H. Brian, Halsall, Joseph H. Nevin, Arthur J. Helmicki H. Thurman Henderson and Chong H. Ahn, An integrated microfluidic biochemical detection system for protein analysis with magnetic bead-based sampling capabilities, Lab Chip, 2002, 2, 27-30 ([20]), Maria A. Schwarz and Peter C. Hauser, Recent developments in detection methods for microfabricated analytical devices, Lab on a Chip, 2001, 1, 1-6 ([21]), Jun Yang. Jianlong Zhao. Mengsu Yang, Optical and electrochemical detection techniques for cell-based microfluidic systems, Anal Bioanal Chem (2006) 384: 1259-1268 ([22])).

The optical method may, for example, be a method chosen from the group comprising a method for measuring a fluorescent signal, a method of analysis by surface plasmon resonance (Homola J, “Present and future of surface plasmon resonance biosensors”, Analytical and Bioanalytical Chemistry 377, 528-53 (2003) ([23])), a method comprising the use of optical tweezers (Molloy J E, Padgett M J, “Lights, action: optical tweezers”, Contemp. Phys. 43, 241-258 (2002) ([24])), a method comprising the use of a laser or of white light, this list not being limiting. When the optical method is a method of analysis by surface plasmon resonance, the support may be transparent.

Advantageously, the optical method may be a method chosen from the group comprising: detection of absorption (D. Boyer, P. Tamarat, A. Maali, B. Lounis, M. Orrit, “Photothermal Imaging of Nanometer-Sized Metal Particles Among Scatterers”, Science 297, 1160 (2002) ([25]), D. Lasne, G. A. Blab, S. Berciaud, M. Heine, L. Groc, D. Choquet, L. Cognet, B. Lounis, “Single Nanoparticle Photothermal Tracking (SNaPT) of 5-nm Gold Beads in Live Cells”, Biophys. J. 91, 4598-4604 (2006) ([26]), Arbouet A, Christofilos D, Del Fatti N, Vallee F, Huntzinger J R, Arnaud L, Billaud P, Broyer M, “Direct measurement of the single-metal-cluster optical absorption”, Phys. Rev. Lett. 93, 127401 (2004) ([27]), Muskens O L, Del Fatti N, Vallee F, Huntzinger J R, Billaud P, Broyer M, “Single metal nanoparticle absorption spectroscopy and optical characterization”, Appl. Phys. Lett. 88, 063109 (2006) ([28])), of reflection, of scattering (De Brabander, M., R. Nuydens, G. Geuens, M. Moeremans, and J. de Mey, “The use of submicroscopic gold particles combined with video contrast enhancement as a simple molecular probe for the living cell”. Cell Motil. Cytoskeleton. 6:105-113 (1986) ([29])), of diffraction, of change in refractive index (D. Boyer, P. Tamarat, A. Maali, B. Lounis, M. Orrit, “Photothermal Imaging of Nanometer-Sized Metal Particles Among Scatterers”, Science 297, 1160 (2002) ([30]), D. Lasne, G. A. Blab, S. Berciaud, M. Heine, L. Groc, D. Choquet, L. Cognet, B. Lounis, “Single Nanoparticle Photothermal Tracking (SNaPT) of 5-nm Gold Beads in Live Cells”, Biophys. J. 91, 4598-4604 (2006) ([31]), Homola J. “Present and future of surface plasmon resonance biosensors” ([32])) and/or the use of a fluorescence emitter, of a means of excitation and of a means of detection of fluorescence (M. J. Saxton, K. Jacobson, “Single-Particle Tracking: Applications to Membrane Dynamics”, Annu. Rev. Biophys. Biomol. Struct., 26, 373-99 (1997) ([33])).

In other words, the observation can be carried out by means of an optical method chosen from the group comprising the use of a fluorescence emitter and of a means of detection of fluorescence, detection of absorption, detection of reflection, detection of scattering and detection of diffraction.

In the case of detection of the movement of the particle by means of a fluorescence method, the fluorescence emitter can be the particle itself or a label associated with the particle and/or with at least one molecule chosen from the first molecule and the second molecule. Preferably, the fluorescence emitter can be the particle itself or a label associated with the particle and/or the second molecule. The fluorescence can be expressed via one or more emission wavelengths. Advantageously, the fluorescence emitter can be any type of molecule known to those skilled in the art, which has the property of absorbing light energy and rapidly releasing it in the form of fluorescence. This emitter may be an organic fluorophore or an inorganic particle. It may, for example, be an emitter chosen from Quantum Dots, nanoparticles doped with lanthanide ions, nanodiamonds, organic fluorophores such as rhodamine, FITC (fluorescein), cyanines, Alexa, dopamine or chromomycin A, fluorescent proteins (such as GFP, YGF, DsRed), and inorganic particles containing organic fluorophores.

When the fluorescence emitter is not the particle itself, the labeling of the particle or of the second molecule with the fluorescence emitter can be carried out by any technique known to those skilled in the art. This may, for example, the labeling techniques described in the documents Chen et al. Nano Letters, vol. 7, No. 3, 690-696, 2007 ([33]), Bek et al., Nano Letters, vol. 8, No. 2, 485-490, 2008 ([34]), WO2007036544 ([35]) or Aslan et al., Current Opinion in Chemical Biology, 9:538-544, 2005 ([36]).

Advantageously, the fluorescence can make it possible to obtain a high signal/noise ratio.

The position of the particle can be visualized by imaging, for example by means of a wide-field microscope, for example commercially available from Zeiss, Olympus or Nikon. It may be equipped with a CCD or EM-CCD or CMOS camera, for example those commercially available from Princeton Instruments, Andor or Hamamatsu. The position can also be visualized by means of a method which has an axial resolution, such as two-photon excitation or nonlinear excitation (W. R. Zipfel, R. M. Williams, W. W. Webb, Nonlinear magic: multiphoton microscopy in the biosciences, Nat. Biotech. 21, 1369 (2003) ([37])) or total internal reflection (TIRF) (A. L. Stout and D. Axelrod, Evanescent field excitation of fluorescence by epi-illumination microscopy, Appl. Opt. 28, 5237 (1989) ([38]), N. L. Thompson and B. C. Lagerholm, Total internal reflection fluorescence: applications in cellular biophysics, Curr. Opin. Biotech., 8, 58-64 (1997) ([39])).

The position of the particle can be determined by means of the adjustment of the image with a two-dimensional Gaussian which gives the center of the particle, or with any other technique, such as correlation between images, used in the field of tracking single molecules/particles (M. K. Cheezum, W. F. Walker, and W. H. Guilford, Quantitative Comparison of Algorithms for Tracking Single Fluorescent Particles, Biophys. J. 81 2378-2388 (2001) ([40])). Accuracy greater than the resolution of an optical microscope, which is limited by light diffraction, can be obtained and depends on the signal/noise ratio and on the characteristics of the detection system (response function, pixel size of the camera) (R. E. Thompson, D. R. Larson, and W. W. Webb, Precise Nanometer Localization Analysis for Individual Fluorescent Probes, Biophysical J. 82 2775-2783 (2002), ([41]), R. J. Ober, S. Ram, and E. S. Ward, Localization Accuracy in Single-Molecule Microscopy Biophysical J. 86, 1185-1200 (2004) ([42])).

The analysis of the interaction according to the movement observed and to the applied flow can be carried out by any means known to those skilled in the art.

The analysis of the interaction can be qualitative or quantitative. It can, for example, make it possible to measure the affinity between the first and the second molecule. It can, for example, make it possible to measure the association and/or dissociation constant of the first and of the second molecule.

For example, the measurement of the dissociation constant between the first and the second molecule under the application of a force F, k_{off}(F), can be obtained quantitatively by measuring the detachment time, on a significant set of molecules—for example 30. Advantageously, this measurement gives the percentage of detached particles as a function of time, which follows a characteristic exponential time law 1/k_{off}(F). Thus, k_{off}(F) can be measured for two (or more) values of the force F.

The dissociation constant in the absence of external force, k_{off}(0), can be determined using the Kramers model (Kramers, H. A., “Brownian motion in a field of force and the diffusion model of chemical reactions” Physica (Amsterdam) 7, 284 (1940) ([43])): k_{off}(0)=w exp(−ΔF/k_{B}T), where w is a prefactor, ΔE the energy barrier to be surmounted for the dissociation, k_{B} the Boltzmann constant and T the temperature of the experiment. When a force F is applied, the energy barrier is reduced by an energy F·a, where a is the characteristic distance of the energy well (which does not need to be known). The new dissociation constant is then (Bell, G. I., Models for the specific adhesion of cells to cells. Science 200, 618-627 (1978) ([44])):

k_{off}(F)=wexp(−ΔE/k_{B}T)exp(Fa/k_{B}T)=k_{off}(0)exp(Fa/k_{B}T).

Thus, on the basis of the dissociation constants obtained experimentally for at least two forces, F₁ and F₂, it is possible to determine the dissociation constant in the absence of force, k_{off}(0), without knowing the distance a. Variants of this approach have been proposed and can also be used (Evan Evans and Ken Ritchie, Dynamic Strength of Molecular Adhesion Bonds, Biophys. J. 72 1541-1555 (1997) ([45])).

For example, the measurement of the equilibrium constant between the first and the second molecule can be carried out by injecting a known amount of second molecule attached to the particle, while waiting a long time considering both the attachment time and the detachment time. By rinsing at low speed with a solution without second molecule and without nanoparticle, and by determining the fraction of particles remaining attached, the concentration attached at equilibrium, and therefore the concentration detached, are obtained. The ratio of the attached concentration to the detached concentration gives the equilibrium constant of the reaction. If the waiting time is short considering the detachment time, the same method gives a measurement of the attachment constant: the amount of particles attached increases exponentially with time.

The method of analysis of the present invention may comprise the detection of the particles bonded to a second molecule not having interacted with the first molecule. The device can, in this case, comprise a means for detecting the particles or the second molecules at the outlet of the channel, i.e. at an end at which the flow arrives. This detection can be based on the physical properties (absorption, reflection or scattering of light, fluorescence, magnetic or electrical properties) and/or chemical properties (for example, interaction with another reactant for an assay) of the particles or of the second molecules. This detection may be advantageous in the context of a method of screening for molecules during which certain second molecules bind to the first molecules, while others do not bind.

Another subject of the present invention is the use of the method or of the device in screening for a candidate molecule for developing a medicament.

The method or the device of the present invention can also be used for parallel, high-throughput measurements of affinity of molecules with one another.

The method or the device of the present invention can also be used in searching for interactions between cells or between a cell and a molecule.

The method or the device of the present invention can be used in evaluating the mobility of particles bonded either to one another or to a support.

The method or the device of the present invention can be used in evaluating the method of movement of bonded particles.

The method or the device of the present invention can be used in evaluating the stretching, the elasticity and the folding of a polymer, of a nucleic acid molecule or of a protein.

The method or the device of the present invention can also be used in evaluating the properties of a cell membrane. In this case, the first molecule may be a cell or the cell membrane, and the second molecule may be a membrane molecule or membrane protein of the cell.

Other advantages may further emerge to those skilled in the art on reading the example below, illustrated by means of the appended figures, given by way of illustration.

### EXAMPLES

### Example 1: Study of Cell Membrane Properties

### 1) Characteristics of the Particles and Coupling to a Peptide Toxin

Nanoparticles (NPs) of yttrium vanadate doped with europium ions, Y_{0.6}Eu_{0.4}VO₄, between 20 and 80 nm in size, are obtained by coprecipitation of Y(NO₃)₃, Eu(NO₃)₃ and Na₃VO₄, as described in the document Huignard et al. ([13]). These nanoparticles are fluorescent: the europium ions inside these nanoparticles can be excited at 466 nm and emit a fluorescence centered on 617 nm.

The particle is coupled to a peptide toxin via a homobifunctional crosslinker, bis(sulfosuccinimidyl)suberate (BS₃). The coupling method is the following and is described in greater detail by Casanova et al. (D. Casanova et al., J. Am. Chem. Soc. 129, 12592 (2007) ([46])):
i) selection of the nanoparticles by size, by centrifugation;
ii) transfer of the NPs from the aqueous solvent to the solvent dimethyl sulfoxide (DMSO);
iii) first acylation reaction between the nanoparticles and the BS₃ crosslinker;
iv) transfer of the NPs from the DMSO to the aqueous solvent and second reaction between nanoparticles-BS₃ and proteins;
v) separation by centrifugation between free proteins and protein-coupled nanoparticles.

The protein selected is the epsilon toxin produced by the anaerobic bacterium Clostridium perfringens types B and D. The toxin binds to a specific receptor which is located in detergent-resistant domains in the membranes of MDCK cells (S. Miyata, J. Minami, E. Tamai, O. Matsushita, S. Shimamoto, A. Okabe, J. Biol. Chem. 277, 39463 (2002) ([47]), C. Chassin et al., Am. J. Pathol. Renal Physiol. 293, F927(2007) ([48])).

When it is desired to attach a single molecule to the nanoparticle and when the reaction of step iv) has an efficiency close to 100%, it is necessary to select a ratio of the concentrations of nanoparticles and of molecules of interest, in this case the epsilon toxin, close to 1:1 for carrying out step iv). The concentration of the BS₃-coupled nanoparticles and of the biomolecules before reaction can be determined via their absorption. After reaction, since the absorption of the biomolecules and of the nanoparticles superimposes, the concentration of the proteins can be determined by means of standard tests such as the Bradford test.

If the efficiency of the reaction of step iv) proves to be lower than 100%, the ratio of the concentrations of the nanoparticles and of the molecules of interest may be adjusted accordingly. The number of molecules bonded to the nanoparticle will follow a Poisson distribution. There will thus be nanoparticles with no bonded molecule and also nanoparticles bonded to one or two molecules and also a small number of nanoparticles bonded to more than two molecules.

The nanoparticles with no bonded molecule will not interact with the membrane receptors and will not disrupt the measurement. In the case of the nanoparticles bonded to two or three molecules, and given the size of the nanoparticles (20-80 nm), the probability of these molecules being arranged so as to be able to interact with more than one receptor is negligible.

### 2) Characteristics of the Microchannel

A microchannel made of polydimethylsiloxane (PDMS) is prepared by soft lithography ([15]).

The dimensions of the microchannel are 50 μm in height, 500 μm in width and 1.5 cm in length. The microchannel has a Y-shaped geometry, and has two inlets: one for the injection of the nanoparticles coupled to the peptide toxin, and the other for generating the predetermined liquid flow.

The microchannel also has an outlet, connected to a reservoir. The reservoir makes it possible to modify the direction of the flow without the risk of introducing bubbles into the microchannel.

3) Injection of the Cells into the Microchannel

A concentrated solution of MDCK cells (˜6×10⁶ cells per ml) in their culture medium (DMEM+10% fetal calf serum (FCS)+1% penicillin-streptomycin) is injected into the microchannel. The cells are then incubated at 37° C. and 5% CO₂ for one day, such that the cells attach to the surface of the microchannel and divide therein.

The formation of air bubbles must be avoided, since bubbles passing through the microchannel might detach the cells or degrade the surface. To do this, the microchannel is completely filled with culture medium. The filling of the microchannel with the culture medium one day before the injection of the cells also makes it possible to remove the air present in the microchannel.

4) Injection of the Nanoparticle-Coupled Peptide Toxins into the Microchannel

The peptide toxin-coupled nanoparticles are suspended with a concentration of 0.05 mM of vanadate ions in a solution of minimal medium (HBSS+10 mM HEPES+1% FCS). The nanoparticles are injected on a microscope using a syringe driver. An incubation is carried out for 5-30 minutes in order for the particle-coupled toxins to bind to the cells, during which time no flow is applied.

It is necessary, at this stage, to verify that these manipulations have not degraded the cells and that they are still attached to the surface of the microchannel, by means of white light transmission images and by observing the fluorescent signal of the cells. It is also useful to verify that the particles have indeed reached the region of the microchannel at which the cells are attached, by fluorescence microscopy.

### 5) Application of the Flow in the Microchannel and Determination of the Applied Force

The manipulation of the sample with the flow is carried out on a microscope. The predetermined flow is applied in the microchannel. The predetermined flow is produced by the injection, using a syringe driver, of a selected volume of fluid per unit time. The fluid used is minimal medium (HBSS+10 mM HEPES+1% FCS). This fluid injection method creates a flow of Poiseuille type in the microchannel. The flowing of the fluid is parallel to the walls everywhere, the speed of the fluid is zero at the walls and the pressure does not vary within the thickness of the flowing.

During the experiment, the flow is varied between 0 and 200 μl/min. The drag force which acts on the particle is determined by the Stokes' relationship:

F=6πμRv

where μ represents the viscosity of the fluid, R the hydrodynamic radius of the particle and v the speed of the fluid around the particle. This relationship is an approximation for channels of infinite length. Since the particle is located at a distance from the cell that is comparable to its hydrodynamic radius, a corrective factor should be applied to the formula above ([16], [17], [18], [19]).

The viscosity of the fluid is determined by measuring the time it takes to flow through a capillary at the ends of which a pressure difference is applied ([14]). A viscosity of 10⁻³ Ns/m² at 20° C. was measured. The radius of each particle is determined by the number of photons emitted per unit time, by employing the method described in Casanova et al. ([46]). For spherical particles, the hydrodynamic radius is very close to the value thus determined.

For a flow rate of 10 μl/min, the average speed of the fluid is 7 mm/s. According to Poiseuille's law, the speed of the fluid has a parabolic profile with a maximum at the center of the channel. For a flow rate of 10 μl/min, the maximum speed at the center of the channel is 10 mm/s. These speeds do not damage the microchannel and do not cause any detachment of the cells from the surface of the microchannel. Furthermore, controlled experiments made it possible to determine that these flow rates do not bring about any movement of the cells nor any modification of the distribution of the microtubules inside the cell nor any modification of the distribution of the lipid rafts in the cell membrane.

The speed of the fluid around the particles was determined by means of the technique of monitoring the objects floating in the flow. In this case, the monitoring of fluorescent Y_{0.6}Eu_{0.4}VO₄ nanoparticles was used.

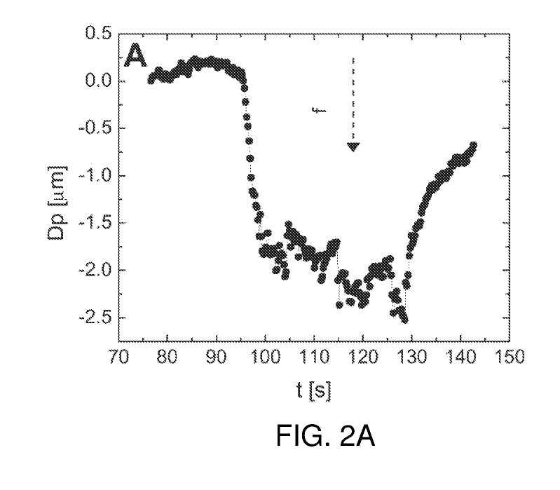

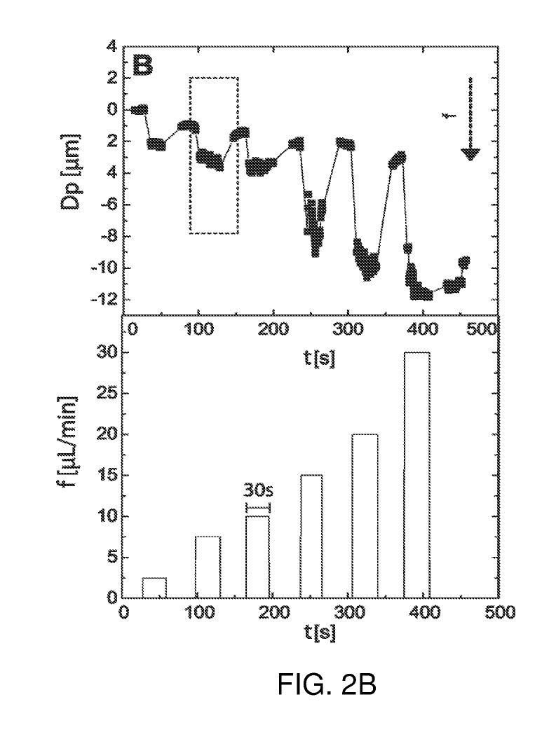

For this study, the flow is applied in a single direction, at flow rates ranging from 0 to 200 μl/min. The value of the flow rate is increased in steps so as to measure the movement of the particle for various values of the flow rate and therefore of the force. Each value of the flow rate is applied for approximately 30 seconds, then the flow is stopped for approximately 30 seconds, then the new flow rate value is applied (cf. FIG. 2B (bottom)).

In the case of our nanoparticles, the force applied can vary between 0 and a few pN (piconewtons) per receptor.

### 6) Recording of Images of Fluorescence Emitted by the Particles

The particles are monitored by monitoring the fluorescence signal emitted by the particles by wide-field fluorescence microscopy. The nanoparticles are excited with an argon laser at 465.8 nm using a 530DCXR dichroic mirror (Chroma). The laser is focused with a microscope objective (63×, NA=1.4 oil immersion objective (Zeiss)). The mid-height width of the excitation zone is 82 μm. The fluorescence emitted by the particles is collected using the same objective and filtered using a 617/8M bandwidth filter (Chroma). The fluorescence images are recorded with an EM-CCD camera (Roper Scientific QuantEM:512SC). The acquisition time per image is 200 ms.

### 7) Image Analysis and Monitoring of Particle Movement

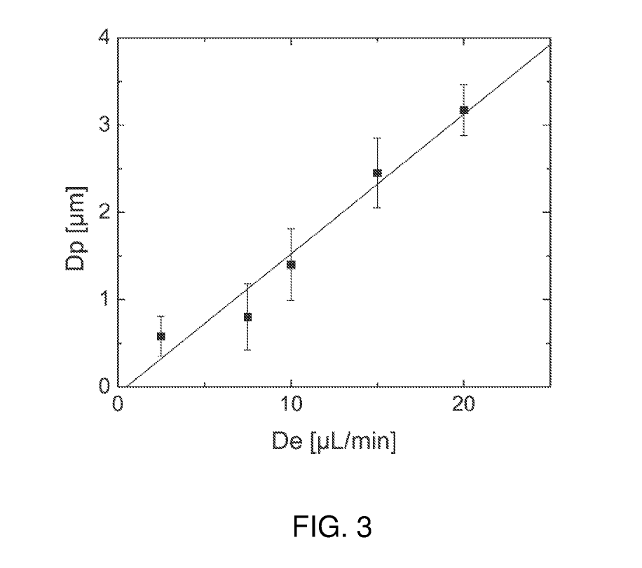

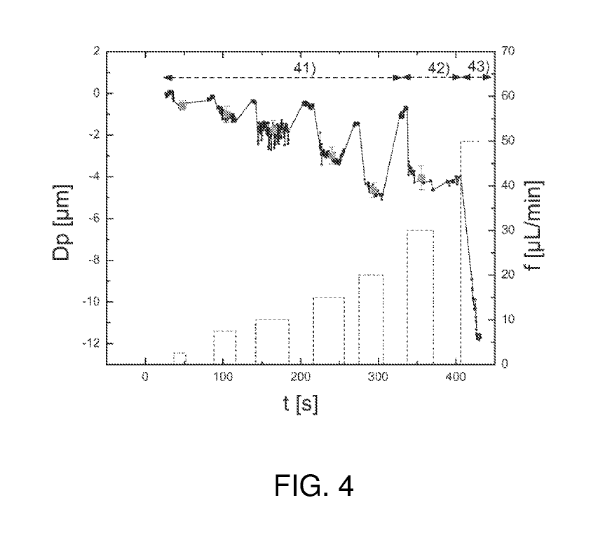

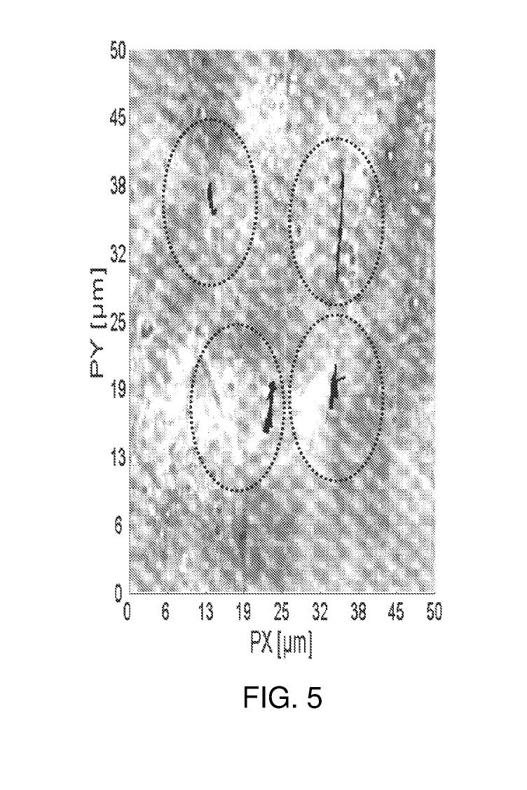

The images are filtered with a low-pass filter and a Laplace filter, to remove the background noise. After a thresholding step, the position of the particles is determined by adjustment with a 2D Gaussian, which makes it possible to provide the center of the particle emission spot with a precision greater than the limit imposed by the microscope resolution. The successive positions of the particle that are obtained from images successively recorded make it possible to measure its movement. Following the application of a constant flow rate, the particle moves and reaches a new equilibrium position after a few seconds. The movement of the particle in FIGS. 2A and 2B (top) is indicated relative to its initial position before application of the flow. To obtain the average movement, the average value of all the positions measured at equilibrium after application of a given flow rate is calculated. The difference between this average value and the initial position before application of the flow is called the average movement. This average movement is shown in FIG. 3 as a function of the applied force calculated for various flow rate values.

### LIST OF REFERENCES

1. Piehler et al., “New methodologies for measuring protein interactions in vivo and in vitro”, Curr Opin Struct Biol. 2005 February; 15(1):4-14.
2. Cooper M A, “Advances in membrane receptor screening and analysis”, J Mol Recognit. 2004 July-August; 17(4):286-315.
3. S. Chu, Laser Manipulation of Atoms and Particles, Science 253, 861 (1991).
4. A. Pralle, P. Keller, E.-L. Florin, K. Simons, and J. K. H. Hörber, Sphingolipid-Cholesterol Rafts Diffuse as Small Entities in the Plasma Membrane of Mammalian Cells, J. Cell Biol. 148, 997-1007 (2000).
5. Kenichi Suzuki, Ronald E. Sterba, and Michael P. Sheetz, Outer Membrane Monolayer Domains from Two-Dimensional Surface Scanning Resistance Measurements, Biophys. J. 79 448-459 (2000).
6. Moy, V. T., Florin, E.-L. & Gaub, H. E. Intermolecular forces and energies between ligands and receptors. Science 264, 257±259 (1994).
7. Hinterdorfer, P., Baumgartner, W., Gruber, H. J., Schilcher, K. & Schindler, H. Detection and localization of individual antibody-antigen recognition events by atomic force microscopy. Proc. Natl Acad. Sci. USA 93, 3477-3481 (1996).
8. Evans, E., Ritchie, K. & Merkel, R. Sensitive force technique to probe molecular adhesion and structural linkages at biological interfaces. Biophys. J. 68, 2580-2587 (1995).
9. R. Merkel, P. Nassoy, A. Leung, K. Ritchie, E. Evans, Nature 397, 50-53 (1999).
10. WO 2009098272.
11. WO 2006009398.
12. I. Barbulovic-Nad, M. Lucente, Y. Sun, M. Zhang, A. R. Wheeler, M. Bussmann, Bio-Microarray Fabrication Techniques—A Review, Critical Reviews in Biotechnology, 26:237-259, 2006.
13. Huignard, A.; Gacoin, T.; Boilot, J.-P. Chem. Mat. 2000, 12, 1090.
14. P. Cousson C. Ancey, Rhéophysique des pätes et des suspensions [Rheophysics of pastes and suspensions], publisher EDP Sciences (1999).
15. P. Tabelling, Introduction à la microfluidique [Introduction to microfluidics], Editions Belin (2003), p. 178-202.
16. A. J. Goldman, R. G. Cox, H. Brenner, Slow viscous motion of a sphere parallel to a plane wall—I Motion through a quiescent fluid, Chem. Eng. Sci. 22 637-651 (1967).
17. A. J. Goldman, R. G. Cox, H. Brenner, Slow viscous motion of a sphere parallel to a plane wall—II Couette flow Chem. Eng. Sci. 22 653-660 (1967).
18. E. Lauga, T. M. Squires, Brownian motion near a partial-slip boundary: A local probe of the no-slip condition, Phys. Fluids 17, 103102 (2005).
19. J. Happel and H. Brenner, Low Reynolds Number Hydrodynamics, Martinus Nijhoff, The Hague (1983), p. 286-340.
20. Jin-Woo Choi, Kwang W. Oh, Jennifer H. Thomas, William R. Heineman, H. Brian, Halsall, Joseph H. Nevin, Arthur J. Helmicki H. Thurman Henderson and Chong H. Ahn, An integrated microfluidic biochemical detection system for protein analysis with magnetic bead-based sampling capabilities, Lab Chip, 2002, 2, 27-30.
21. Maria A. Schwarz and Peter C. Hauser, Recent developments in detection methods for microfabricated analytical devices, Lab on a Chip, 2001, 1, 1-6.
22. Jun Yang, Jianlong Zhao, Mengsu Yang, Optical and electrochemical detection techniques for cell-based microfluidic systems, Anal Bioanal Chem (2006)384:1259-1268.
23. Homola J. “Present and future of surface plasmon resonance biosensors”, ANALYTICAL AND BIOANALYTICAL CHEMISTRY 377, 528-53 (2003).
24. Molloy J E, Padgett M J, “Lights, action: optical tweezers”, CONTEMP. PHYS. 43, 241-258 (2002).
25. D. Boyer, P. Tamarat, A. Maali, B. Lounis, M. Orrit, “Photothermal Imaging of Nanometer-Sized Metal Particles Among Scatterers”, Science 297, 1160 (2002).
26. D. Lasne, G. A. Blab, S. Berciaud, M. Heine, L. Groc, D. Choquet, L. Cognet, B. Lounis, “Single Nanoparticle Photothermal Tracking (SNaPT) of 5-nm Gold Beads in Live Cells”, Biophys. J. 91, 4598-4604 (2006).
27. Arbouet A, Christofilos D, Del Fatti N, Valle F, Huntzinger J R, Arnaud L, Billaud P, Broyer M, “Direct measurement of the single-metal-cluster optical absorption”, Phys. Rev. Lett. 93, 127401 (2004).
28. Muskens O L, Del Fatti N, Vallee F, Huntzinger J R, Billaud P, Broyer M, “Single metal nanoparticle absorption spectroscopy and optical characterization”, Appl. Phys. Lett. 88, 063109 (2006).
29. De Brabander, M., R. Nuydens, G. Geuens, M. Moeremans, and J. de Mey, “The ue of submicroscopic gold particles combined with video contrast enhancement as a simple molecular probe for the living cell”. Cell Motil. Cytoskeleton. 6:105-113 (1986).
30. D. Boyer, P. Tamarat, A. Maali, B. Lounis, M. Orrit, “Photothermal Imaging of Nanometer-Sized Metal Particles Among Scatterers”, Science 297, 1160 (2002).
31. D. Lasne, G. A. Blab, S. Berciaud, M. Heine, L. Groc, D. Choquet, L. Cognet, B. Lounis, “Single Nanoparticle Photothermal Tracking (SNaPT) of 5-nm Gold Beads in Live Cells”, Biophys. J. 91, 4598-4604 (2006).
32. Homola J, “Present and future of surface plasmon resonance biosensors”. Anal Bioanal Chem, vol. 377, 528-539, 2003.
33. Chen et al. Nano Letters, vol. 7, No. 3, 690-696, 2007.
34. Bek et al., Nano Letters, vol. 8, No. 2, 485-490, 2008.
35. WO2007036544.
36. Aslan et al., Current Opinion in Chemical Biology, 9:538-544, 2005.
37. W. R. Zipfel, R. M. Williams, W. W. Webb, Nonlinear magic: multiphoton microscopy in the biosciences, Nat. Biotech. 21, 1369 (2003).
38. A. L. Stout and D. Axelrod, Evanescent field excitation of fluorescence by epi-illumination microscopy, Appl. Opt. 28, 5237 (1989).
39. N. L. Thompson and B. C. Lagerholm, Total internal reflection fluorescence: applications in cellular biophysics, Curr. Opin. Biotech., 8, 58-64 (1997).
40. M. K. Cheezum, W. F. Walker, and W. H. Guilford, Quantitative Comparison of Algorithms for Tracking Single Fluorescent Particles, Biophys. J. 81 2378-2388 (2001).
41. R. E. Thompson, D. R. Larson, and W. W. Webb, Precise Nanometer Localization Analysis for Individual Fluorescent Probes, Biophysical J. 82 2775-2783 (2002).
42. R. J. Ober, S. Ram, and E. S. Ward, Localization Accuracy in Single-Molecule Microscopy Biophysical J. 86, 1185-1200 (2004).
43. Kramers, H. A., “Brownian motion in a field of force and the diffusion model of chemical reactions” Physica (Amsterdam) 7, 284 (1940).
44. Bell, G. I., Models for the specific adhesion of cells to cells. Science 200, 618-627 (1978).
45. Evan Evans and Ken Ritchie, Dynamic Strength of Molecular Adhesion Bonds, Biophys. J. 72 1541-1555 (1997).
46. Casanova et al., Appl. Phys. Lett. 89, 253103 (2006).
47. S. Miyata, J. Minami, E. Tamai, O. Matsushita, S. Shimamoto, A. Okabe, J. Biol. Chem. 277, 39463 (2002).
48. C. Chassin et al., Am. J. Pathol. Renal Physiol. 293, F927 (2007).
49. Schmidt Brian J. et al. “Catch Strip Assay for the Relative Assessment of Two-Dimensional Protein Association Kinetics”, Anal. Chem. 2008, 80:944-950.

1. A method for analyzing an interaction between a first molecule and a second molecule bonded to a particle, comprising the following steps:
bringing the first molecule and the second molecule bonded to the particle into contact under conditions enabling the interaction thereof,
applying a predetermined liquid flow to the particle bonded to the second molecule,
observing a movement of the particle bonded to the second molecule caused by the applied flow,
analyzing the interaction according to the movement observed and to the applied flow,
the particle having a greater hydrodynamic resistance than the first and/or second molecule, and having a mass Péclet number greater than 1, and having a nanometric size dimension between 1 and 100 nm.

2. The method according to claim 1, wherein the movement of the particle is observed by means of the physical and/or chemical properties of the particle.

3. The method according to claim 2, wherein the observation is carried out by a method selected from the group comprising an optical method, a chemical method, an electrochemical method and a magnetic method.

4. The method according to claim 3, wherein the observation is carried out by an optical method selected from the group comprising the use of a fluorescence emitter and of a means for detecting fluorescence, detection of absorption, detection of reflection, detection of scattering and detection of diffraction.

5. The method according to claim 4, wherein the fluorescence emitter is the particle itself or a label associated with the particle and/or with at least one molecule selected from the first molecule and the seco(Original) nd molecule.

6. The method according to claim 1, wherein the predetermined liquid flow is such that it makes it possible to apply to the particle a force F equal to 6πμRv, wherein μ represents the viscosity of the fluid, R the hydrodynamic radius of the particle and v the speed of the fluid around the particle.

7. The method according to claim 1, wherein the analysis is qualitative or quantitative.

8. The method according to claim 1, wherein the interaction is an interaction between biological molecules or between a biological molecule and a chemical molecule.

9. The method according to claim 1, wherein the first molecule is a molecule of a cell membrane.

10. The method according to claim 3, wherein the optical method comprises surface plasmon resonance.

11. The method according to claim 1, wherein the movement of the particle bonded to the second molecule caused by the applied flow is due to the breaking of the interaction between the first and the second molecule.

12. The method according to claim 7, wherein the movement of the particle bonded to the second molecule caused by the applied flow is due to the breaking of the interaction between the first and the second molecule.

13. A method of screening for a candidate molecule for developing a medicament using a method according to claim 1.

14. The method according to claim 1, wherein the movement of the particle bonded to the second molecule is a change of spatial position of said particle bonded to the second molecule caused by the applied flow, relative to the moment at which the first and second molecules interact in the absence of applied flow.

15. The method according to claim 1, wherein the interaction between the first and second molecules is a specific or non-specific noncovalent and reversible bond.

16. The method according to claim 15, wherein the non-specific interaction is an interaction between a biological molecule and a surface.

17. The method according to claim 1, wherein the interaction between the first and second molecules is a specific interaction due to electrostatic force, hydrophobic bonds, hydrogen bonds and/or Van der Waals force.

18. The method according to claim 17, wherein the specific interaction is antigen-antibody, ligand-receptor or enzyme-substrate interaction.

19. The method according to claim 1, where the analysis of the interaction is the determination of the dissociation constant between the first and the second molecule in the absence of external force.

20. The method according to claim 19, wherein the dissociation constant is determined by observing the movement of the particle bonded to the second molecule caused by two or more applied flow values.

21. The method according to claim 1, wherein the first molecule, the second molecule, or both the first and second molecule are an organic molecule.

22. The method according to claim 21, wherein the organic molecule is selected from the group comprising an amino acid, a peptide, a protein, a nucleoside, a nucleotide, a nucleic acid, a fatty acid, a simple or complex lipid, a sugar, a monosaccharide, a polysaccharide, a phospholipid, a glycolipid, a glycoprotein, a lipoprotein, a glycolipoprotein, or any other molecule of biological origin which interacts or is capable of interacting with another molecule.

23. The method according to claim 1, wherein the first molecule, second molecule, or the first and second molecule, has a molecular weight ranging from 100 to 300,000 g·mol⁻¹.

24. The method according to claim 24, wherein the particle has at least one dimension which is between five and one hundred times that of the second molecule.

25. The method according to claim 1, wherein the particle has a substantially round or oval shape, or an ovoid, cylindrical or discoid shape.
